(19) United States
(12) Patent Application Publication
(10) Pub. No.: US 2016/0279007 A1
(43) Pub. Date: Sep. 29, 2016

(54) Patient positioning support

(71) Applicant: Gerald J. Flatt, Lutz, FL (US)

(72) Inventor: Gerald J. Flatt, Lutz, FL (US)

(21) Appl. No.: 14/545,091

(22) Filed: Mar. 24, 2015

(51) Int. Cl. A61G 13/12; A61G 13/04; A61F 5/37

(52) U.S. Cl. CPC A61G 13/126; A61G 13/1205; A61F 5/3769; A61G 13/04; A61G 2200/32

(57) ABSTRACT

A patient positioning support to support a patient on a tiltable medical procedures table during a medical procedure comprising a top layer of resilient cushion of foam to engage and support the torso of a patient thereon and a bottom layer of anti-skid foam to engage the tiltable medical procedures table to adhere the patient positioning support thereto and a resilient arm restraint extending from each side of the patient positioning support to cradle and cover the corresponding arm of the patient therein.

### CROSS-REFERENCE

This application claims priority of pending application Ser. No. 61/967,863 filed Mar. 28, 2014.

### BACKGROUND

1. Field of the Invention

A patient positioning support to cushion the torso of a patient on a tiltable medical procedures table during a medical procedure.

2. Description of the Prior Art

Various surgical tables having articulated table tops have been developed to meet the growing demand of surgical tables used with a number of different operations. Among the functions desirable for a surgical table are the ability to raise and lower the patient positioning support surface, provide lateral tilting of the patient positioning support surface to the left and right, provide a pivoting of a seat section independently of a back section, provide pivoting of a leg section independently of the seat section, movement of the patient positioning support surface into the Trendelenburg and reverse Trendelenburg positions, and movement of the patient positioning support surface into the flex and reflex positions.

U.S. Pat. No. 8,511,314 shows a viscoelastic foam pad to assist in holding a patient in a desired position on an inclined support table and to assist in minimizing sliding, shifting, or similar undesirable movements of the patient on the support table. The viscoelastic foam also cushions and supports the patient while promoting a distribution of pressure forces across the patient's body to reduce and/or minimize damage to nerves and/or tissue from concentrated pressure forces.

U.S. Pat. No. 8,464,720 discloses a method of securing a patient on an operating table comprising a viscoelastic pad to support and hold a patient on the table during a medical procedure while the table and patient are in an inclined position to minimization of pressure forces on the patient's body, as well as promote a secure cushioning and holding of the patient in a desired position on the table to minimize injury to the patient.

U.S. Pat. No. 5,754,997 describes a cushion to support a patient over gaps which occur between adjacent sections of a surgical table formed with a soft foam upper layer and a flexible lower layer which is sufficiently rigid to form a support for continuously supporting the weight of a patient across a gap located between adjacent sections of the surgical table.

U.S. Pat. No. 5,138,730 shows a multi-layer cushion support including a top layer of resilient foam having a plurality of hardened projections extending outwardly therefrom.

The Journal of Minimally Invasive Gynecology describes an egg-crate anti-skid material and patient positioning pad to support and prevent patient shifting during robotic assisted gynecologic procedures.

U.S. Pat. No. 6,701,558 shows a patient positioning support including at least one inflatable air bladder formed of anti-shear material and a cover including a foam layer and a highly stretchable material layer.

U.S. Pat. No. 6,578,219 relates to a patient positioning supporting surface for use with a conventional operating room table having at least one pad for supporting a patient having a chamber for containing a quantity of fluid. The fluid contained within the chamber is a deformable and pressure compensating, radiolucent fluid. The chamber and the fluid are cooperatively adapted to maintain the patient in a fixed position while substantially minimizing the interface pressure and shear force between the torso pad 16 and the patient.

U.S. Pat. No. 6,484,334 shows a patient positioning support apparatus including a base, a frame coupled to the base and a mattress supported by the frame and positioned to lie above the frame to support a patient. The mattress has at least one bladder configured to receive a medium.

U.S. Pat. No. 7,731,282 discloses a seating pad assembly for use by travelers to increase the comfort for a seat on a public transportation vehicle or at a public transit terminal. The seating pad assembly includes a cushion formed from a viscoelastic foam and having dimensions substantially conforming to the dimensions of at least the hip/thigh support and the back support of the seat.

PCT/DK91/00136 teaches a cushion of plastic foam comprising a wedge shaped bottom portion which changes impression hardness and carrying capacity at fluctuating temperature and air humidity and of a wedge shaped top portion of a soft more porous plastic foam which form the backwardly inclined front surface shows decreased thickness towards the rear edge of the cushion.

U.S. Pat. No. 6,159,574 describes a laminated support for pressure-relief including an upper layer of visco-elastic foam, a middle layer of visco-elastic foam having a greater hardness and a bottom layer of highly resilient polyurethane foam. These layers are sandwiched between two layers of reticulated filter polyurethane foam.

US 2004/0237206 discloses a pad of visco-elastic foam to provide contouring support. Two layers of foam are used with different densities to allow the user to select the degree to which the foam contours to the body.

U.S. Pat. No. 5,669,094 shows a mattress comprising a top layer of visco-elastic polyurethane foam having a low rebound property and which is temperature sensitive having a thickness of approximately ½ to 1 and ½ inches.

U.S. Pat. No. 5,720,057 describes a portable mattress assembly having mattress support material to provide a suitable balance between portability and comfort.

U.S. Pat. No. 5,906,205 shows a surgical positioning device for supporting the body of a patient in a selected lateral position comprises a bag constructed of flexible, air-impermeable material filled with a charge of elastically deformable expanded polystyrene beads. An inflatable pillow is attached to the upper edges of the bag at the center thereof such that when inflated, the pillow supports the patient's axilla and eliminates pressure, thereby to prevent injury to the patient's brachial plexus.

U.S. Pat. No. 8,539,621 and U.S. Pat. No. 8,539,622 relate to a patient positioner for maintaining a patient's position during a medical procedure comprising a generally rectangular body anchoring portion having a longitudinally disposed head and tail ends and two transversely disposed opposing side ends, placeable atop an operating table. There are a pair of spaced apart chest straps, a pair of spaced apart support base straps, a pair of upper arm straps, and a pair of wrist straps. An extender strap is provided to facilitate securement of the support base straps to a support structure. In use, the body anchoring portion is positioned under the patient's torso with the substrate backing side ends wrapped over the arms while the chest straps are brought over the patient's shoulders and criss-crossed over the patient's chest and secured on an opposing side rail while the upper arm and wrist straps are brought from underneath the patient's torso and wrapped over the patient's arms.

Additional examples of the prior art are found in the following documents: U.S. Pat. No. 3,227,440; U.S. Pat. No. 4,991,242; U.S. Pat. No. 5,362,302; U.S. Pat. No. 5,396,672; U.S. Pat. No. 5,960,496; U.S. Pat. No. 5,966,763; U.S. Pat. No. 6,182,316; U.S. Pat. No. 6,226,820; U.S. Pat. No. 6,256,821; U.S. Pat. No. 6,367,106; U.S. Pat. No. 6,484,334; U.S. Pat. No. 6,568,010; U.S. Pat. No. 6,622,727; U.S. Pat. No. 6,625,830; U.S. Pat. No. 6,754,923; U.S. Pat. No. 6,877,176; U.S. Pat. No. 6,880,189; U.S. Pat. No. 6,924,467; U.S. Pat. No. 6,957,465; U.S. Pat. No. 7,076,822; U.S. Pat. No. 7,082,631; U.S. Pat. No. 7,240,384; U.S. Pat. No. 7,287,289; U.S. Pat. No. 7,424,760; U.S. Pat. No. 7,441,293; U.S. Pat. No. 7,461,894; U.S. Pat. No. 7,555,794; U.S. Pat. No. 7,757,318; U.S. Pat. No. 7,849,540 and U.S. Pat. No. 8,469,911.

While some of the prior art may contain some similarities relating to the present invention, none of them teach, suggest or include all of the advantages and unique features of the invention disclosed hereafter.

### SUMMARY OF THE INVENTION

The present invention relates to a patient positioning support to position and support a patient on a tiltable medical procedures table during a medical procedure. The patient positioning support is particularly useful in robotic, gynecological, colorectal, laparoscopic and urological procedures.

The patient positioning support comprises a torso pad to engage and support the patient on the upper surface of the tiltable medical procedures table comprising a convoluted top cushion and a bottom anti-skid pad or member.

The convoluted top cushion comprises a resilient, flexible base having a plurality of projections and protrusions extending upwardly or outwardly therefrom; while, the bottom anti-skid pad or member comprises a substantially flat flexible layer.

The patient positioning support may further include at least one attachment device attached or affixed to each side of the torso pad to attach or fasten the patient positioning support to the tiltable medical procedures table.

In addition, a pair of patient body straps may be used to further secure the patient and torso pad in position on the tiltable medical procedures table.

The torso pad molds to the patient's body, providing stability and maintains patient position throughout the procedure by preventing patient slippage.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 is a top view of a patient positioned and supported by the patient positioning support of the present invention on a tiltable medical procedures table.

FIG. 2 is a top view of the patient positioning support of the present invention.

FIG. 3 is a partial cross-sectional view of the torso support of the present invention.

FIG. 4 is a top view of the alternate embodiment of the patient positioning support of the present invention.

FIG. 5 is top view of an alternate embodiment of the patient positioning support of the present invention depicted in FIG. 4 with the arm restraints deployed.

FIG. 6 is an exploded view of the alternate embodiment of the patient positioning support of the present invention depicted in FIG. 4.

Similar reference characters refer to similar parts throughout the several views of the drawings.

### DETAILED DESCRIPTION OF THE INVENTION

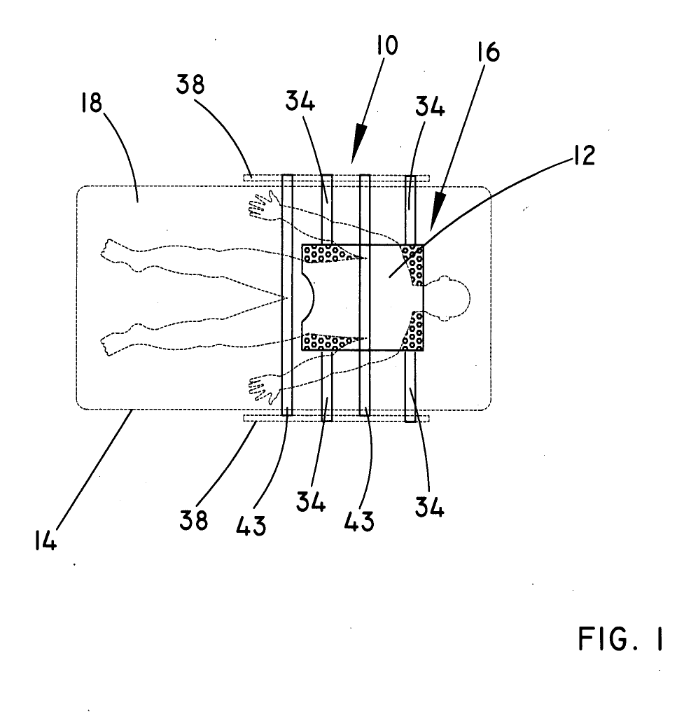

As shown in FIG. 1, the present invention relates to a patient positioning support generally indicated as 10 to position and support a patient 12 on a tiltable medical procedures table generally indicated as 14 during a medical procedure. The patient positioning support 10 comprises a torso pad generally indicated as 16 to engage and support the patient 12 on the upper surface 18 of the tiltable medical procedures table 14.

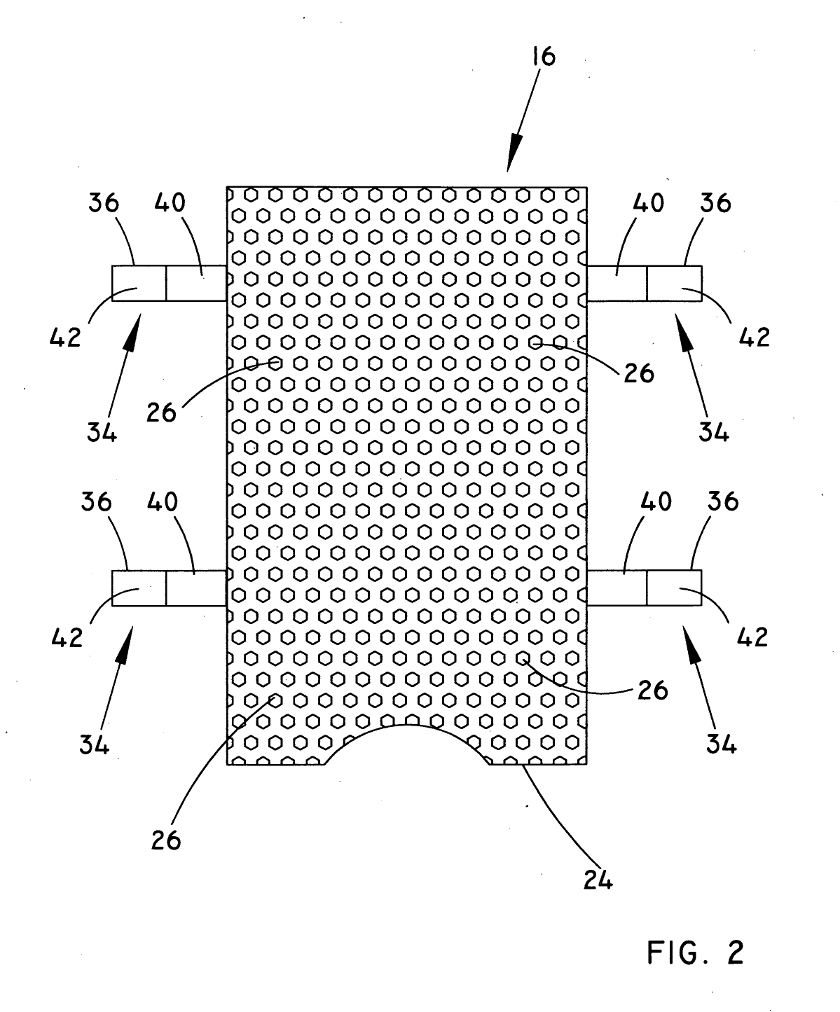

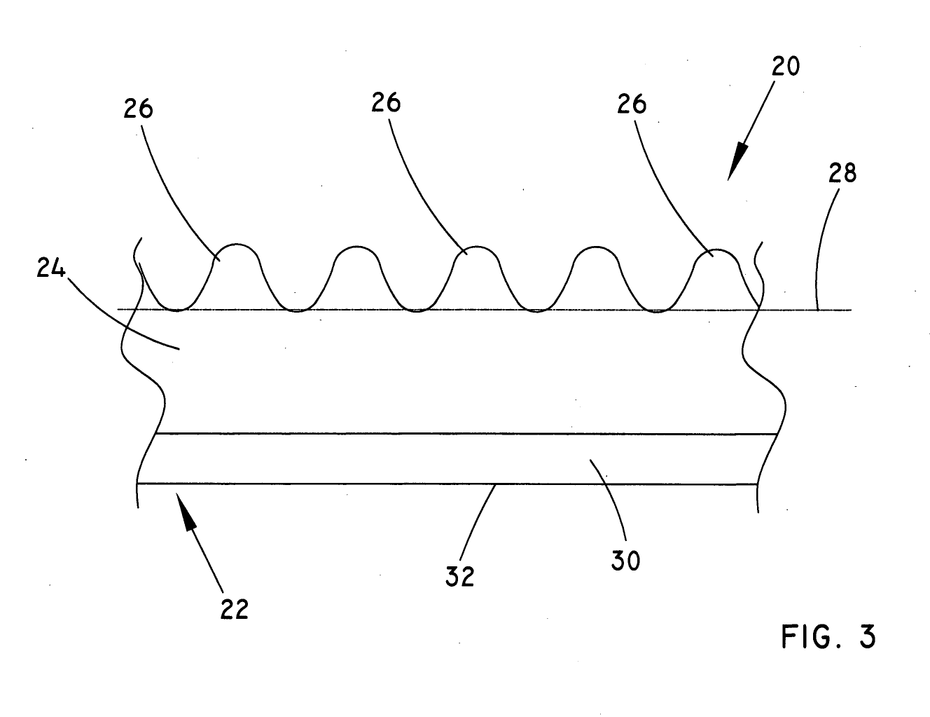

As shown in FIGS. 2 and 3, the torso pad 16 comprises a convoluted top cushion generally indicated as 20 and a bottom anti-skid pad or member generally indicated as 22.

The convoluted top cushion 20 comprises a resilient, flexible base 24 having a plurality of projections and protrusions each indicated as 26 extending upwardly or outwardly therefrom. The resilient, flexible base 24 is about ⅝^{th} of an inch thick having a density of about 1.25 pounds per cubic foot to about 1.35 pounds per cubic foot and an indentation force deflection of about 37 pounds to about 43 pounds at 25 percent (25%) deflection to about 85 pounds to 91 pounds at 65 percent (65%) deflection. Each projection or protrusion 26 extends at least about ½ of an inch from the upper surface 28 of the resilient, flexible base 24. There are about 13 projections or protrusions 26 in a four (4) inch square on the upper surface 28 of the resilient, flexible base 24. The fire resistant rating is about CAL 117.

The convoluted top cushion 20 is preferably constructed of latex-free, polyurethane foam.

The bottom anti-skid pad or member 22 comprises a substantially flat flexible layer 30 about ¼^{th} inch thick having a indentation force deflection of 25 percent (25%) at about 7 pounds per cubic foot to about 18 pounds per cubic foot. The lower surface 32 of the substantially flat flexible layer 30 of the bottom anti-skid pad or member 22 has a coefficient of static friction sufficient to prevent the torso pad 16 from sliding or moving on the upper surface 18 of the tiltable medical procedures table 14.

The anti-skid pad or member 22 is preferably constructed of latex-free, hydrophilic tight cell polyurethane foam.

The patient positioning support 10 further includes at least one attachment device generally indicated as 34 attached or affixed to each side of the torso pad 16 to attach or fasten the patient positioning support 10 to the tiltable medical procedures table 12. Each attachment device 34 may comprise a torso pad strap 36 to be looped or wrapped around rails 38 on each side of the tiltable medical procedures table 14 and held or secured in place by a fastener such as a hook 40 and loop 42 combination. Of course, other state of the art fasteners are suitable. The torso pad 16 straps 36 may be sandwiched between the resilient, flexible base 24 of the convoluted top cushion 20 and the bottom anti-skid pad or member 22 or secured to the bottom anti-skid pad or member 22.

In addition, a pair of patient body straps each indicated as 43 may be used to further secure the patient 12 and torso pad 16 in position on the tiltable medical procedures table 14.

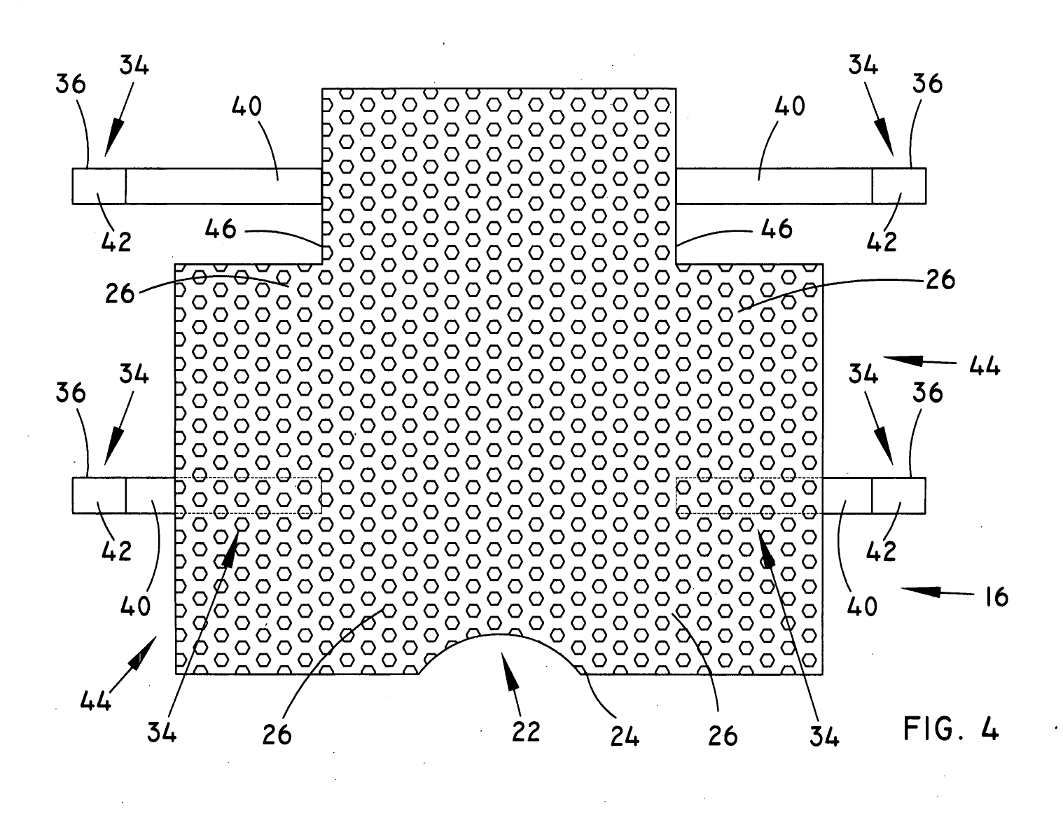

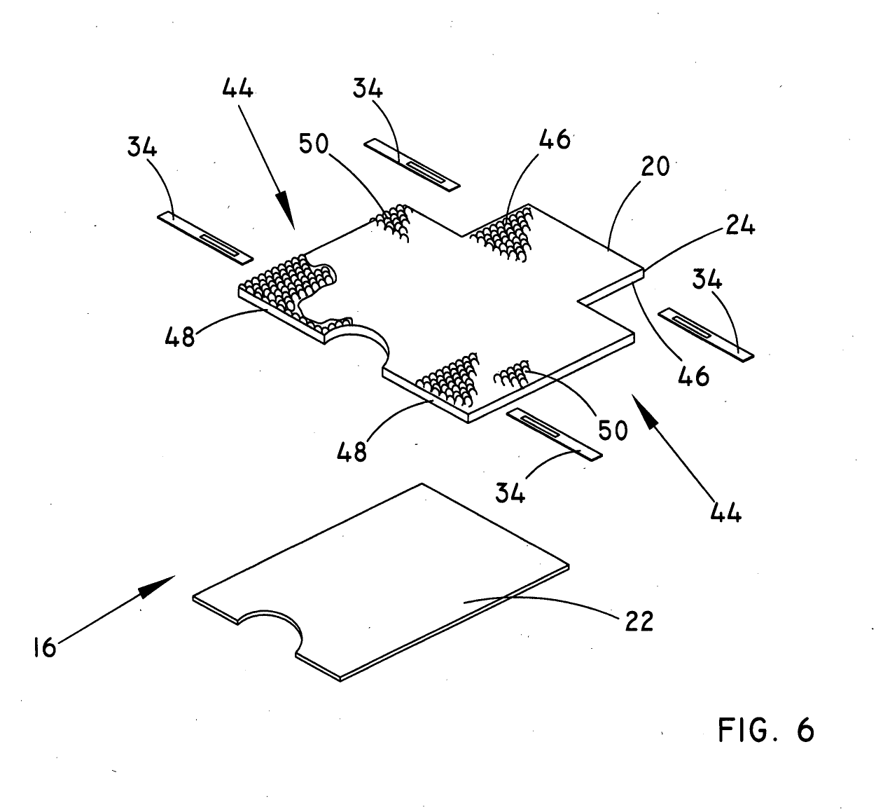

FIGS. 4 through 6 depict an alternate embodiment of the torso pad 16 including a pair of resilient, flexible arm restraints each generally indicated as 44 to cradle and cover the patient's arms. As best shown in FIG. 4, each resilient, flexible arm restraint 44 extends from a corresponding side 46 of the torso pad 16 to wrap and restrain the corresponding arm of the patient 12 therein during a medical procedure. This width of each resilient, flexible arm restraint 44 is about ½ the width of the torso pad 16.

Each resilient, flexible arm restraint 44 comprises a resilient, flexible base 48 having a plurality of resilient projections or protrusions each indicated as 50 extending upwardly or outwardly therefrom.

The resilient, flexible arm restraint 44 may be integrally formed as an extension of the resilient, flexible base 24 of the convoluted top cushion 20 or constructed separately and attached or affixed to each side of the resilient, flexible base 24 of the substantially flat flexible layer 30 or bottom anti-skid pad or member 22.

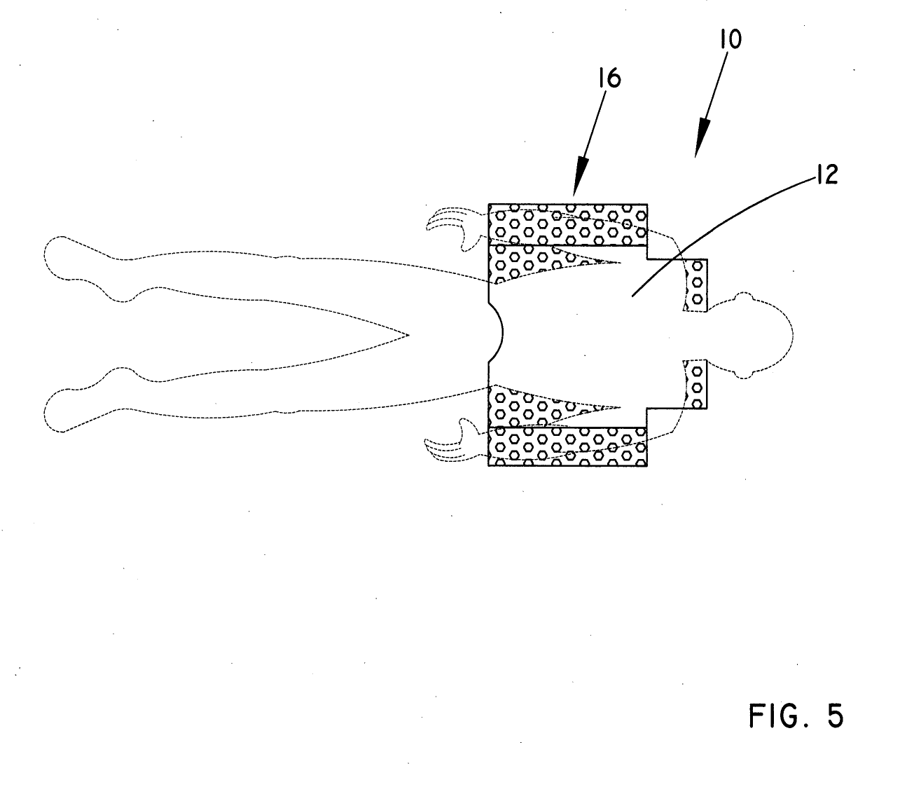

The torso pad 16 depicted in FIGS. 4 and 5 is similar in construction and material as the torso pad 16 depicted in FIGS. 2 and 3. As such, similar components or structural elements are similarly designated. The convoluted top cushion 20 comprises a resilient, flexible base 24 having a plurality of projections and protrusions each indicated as 26 extending upwardly or outwardly therefrom. The resilient, flexible base 24 is about ⅝^{th} of an inch thick having a density of about 1.25 pounds per cubic foot to about 1.35 pounds per cubic foot and an indentation force deflection of about 37 pounds to about 43 pounds at 25 percent (25%) deflection to about 85 pounds to 91 pounds at 65 percent (65%) deflection. Each projection or protrusion 26 extends at least ½ of an inch from the upper surface 28 of the resilient, flexible base 24. There are about 13 projections or protrusions 26 in a four (4) inch square on the upper surface 28 of the resilient, flexible base 24. The fire resistant rating is about CAL 117.

The convoluted top cushion 20 is preferably constructed of latex-free, polyurethane foam.

The bottom anti-skid pad or member 22 comprises a substantially flat flexible layer 30 about ¼^{th} inch thick having a indentation force deflection of 25 percent (25%) at about 7 pounds per cubic foot to about 18 pounds per cubic foot. The lower surface 32 of the substantially flat flexible layer 30 of the bottom anti-skid pad or member 22 has a coefficient of static friction sufficient to prevent the torso pad 16 from sliding or moving on the upper surface 18 of the tiltable medical procedures table 14.

The anti-skid pad or member 22 is preferably constructed of latex-free, hydrophilic tight cell polyurethane foam.

The patent support 10 further includes at least one attachment device generally indicated as 34 attached or affixed to each side of the torso pad 16 to attach or fasten the patient positioning support 10 to the tiltable medical procedures table 12. Each attachment device 34 may comprise a torso pad strap 36 to be looped or wrapped around rails 38 on each side of the tiltable medical procedures table 14 and held or secured in place by a fastener such as a hook 40 and loop 42 combination. Of course, other state of the art fasteners are suitable. The torso pad 16 straps 36 may be sandwiched between the resilient, flexible base 24 and the convoluted top cushion 20 and the bottom anti-skid pad or member 22 or secured to the bottom anti-skid pad or member 22.

In addition, a pair of patient body straps each indicated as 43 may be used to further secure the patient 12 and torso pad 16 in position on the tiltable medical procedures table 14.

Each resilient, flexible arm restraint 44 is about ⅝^{th} inch thick having a density of about 1.25 pounds per cubic foot to about 1.35 pounds per cubic foot and indentation force deflection of about 37 pounds to about 43 pounds at 25 percent (25%) deflection to about 85 pounds to 91 pounds at 65 percent (65%) deflection. Each resilient flexible arm restraint 24 is preferably constructed of latex-free polyurethane foam.

The patient positioning support 10 is intended primarily for use with colorectal, robotic, laparoscopic, gynecological and urological procedures requiring medical procedure tables capable of configurations such as a Trendelenburg, reverse Trendelenburg or tilted position as shown in FIGS. 3 through 6 of U.S. Pat. No. 5,754,997.

In use, after the tiltable medical procedures table 14 is placed in the Lithotomy position.
The torso pad 16 is positioned on the tiltable medical procedures table 14 with torso pad straps 36 facing upward.
Attach the torso pad straps 36 to the rails 38 by looping under the corresponding rail 38 and fastening the hook 40 and loop 42 combination together.
Place the patient 12 on the torso pad 16. Position the patient 12 so that the shoulders do not extend past the upper end of the torso pad 16. Make an effort not to drag the patient 12 on the torso pad 16. Make certain that the torso pad 16 remains completely flat on the tiltable medical procedures table 14 at all times.
Wrap the patient's arms in the resilient, flexible arm restraints 44 using normal hospital protocol for positioning the patient's arms.

The patient 12 and torso pad 16 may be strapped to the tiltable medical procedures table 14 by the patient body straps 43 placed across the patient's body.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

What is claimed is:

1. A patient positioning support to support a patient on a tiltable medical procedures table during a medical procedure comprising a top layer of resilient cushion of foam to engage and support the torso of a patient thereon and a bottom layer of anti-skid foam to engage the tiltable medical procedures table to adhere said patient positioning support thereto.

2. The patient positioning support of claim 1 wherein said top layer of resilient cushion comprises a resilient, flexible base having a plurality of projections or protrusions extending upwardly or outwardly therefrom and said bottom layer of anti-skid foam comprises a substantially flat flexible layer.

3. The patient positioning support of claim 2 wherein said resilient flexible base is about ⅝th of an inch thick and each projection or protrusion extends at least about ½ of an inch from the upper surface of said resilient flexible base.

4. The patient positioning support of claim 3 wherein said resilient flexible base has a density of about 1.25 pounds per cubic foot to about 1.35 pounds per cubic foot and an indentation force deflection of about 37 pounds to about 43 pounds at 25 percent (25%) deflection to about 85 pounds to 91 pounds at 65 percent (65%) deflection.

5. The patient positioning support of claim 4 wherein said resilient flexible base has about 13 projections or protrusions in a four inch square on said upper surface of said resilient flexible base.

6. The patient positioning support of claim 2 wherein said substantially flat flexible layer of said bottom layer of anti-skid foam is about ¼^{th} inch thick having a indentation force deflection of 25 percent (25%) at about 7 pounds per cubic foot to about 18 pounds per cubic foot.

7. The patient positioning support of claim 6 wherein said lower surface of said substantially flat flexible layer of the bottom anti-skid pad or member has a coefficient of static friction sufficient to prevent the torso pad from sliding or moving on the upper surface of the tiltable medical procedures table.

8. The patient positioning support of claim 2 wherein said lower surface of said substantially flat flexible layer of the bottom anti-skid pad or member has a coefficient of static friction sufficient to prevent the torso pad from sliding or moving on the upper surface of the tiltable medical procedures table.

9. The patient positioning support of claim 2 wherein said bottom layer of anti-skid foam is preferably constructed of latex-free, hydrophilic tight cell polyurethane foam.

10. The patient positioning support of claim 1 wherein said patient positioning support further includes at least one attachment device attached or affixed to each side of said torso pad to attach or fasten said patient positioning support to the tiltable medical procedures table.

11. The patient positioning support of claim 10 wherein each said attachment device comprises a torso pad strap to be looped or wrapped around rails on each side of the tiltable medical procedures table and held or secured in place by a fastener.

12. The patient positioning support of claim 10 further includes a pair of patient body straps to further secure the patient and said torso pad in position on the tiltable medical procedures table.

13. The patient positioning support of claim 1 further includes a resilient flexible arm restraint extending from each side of said torso pad to cradle and cover the corresponding arm of the patient therein.

14. The patient positioning support of claim 13 wherein each said resilient, flexible arm restraint comprises a resilient, flexible base having a plurality of resilient projections or protrusions extending upwardly or outwardly therefrom.

15. The patient positioning support of claim 13 wherein each said resilient, flexible arm restraint is integrally formed as an extension of said top layer of resilient cushion of foam.

16. A patient positioning support to support a patient on a tiltable medical procedures table during a medical procedure comprising a torso pad to engage and support the torso of a patient thereon and a resilient, flexible arm restraint extending from each side of said torso pad to cradle and cover the corresponding arm of the patient therein.

17. The patient positioning support of claim 16 wherein said torso pad comprises a top cushion comprises a resilient, flexible base having a plurality of projections and protrusions extending upwardly or outwardly and each said resilient, flexible arm restraint comprises a resilient, flexible base having a plurality of resilient projections or protrusions extending upwardly or outwardly therefrom.

18. The patient positioning support of claim 17 wherein said resilient, flexible arm restraint is integrally formed as an extension of said resilient, flexible base of said top cushion.

19. The patient positioning support of claim 18 wherein said torso pad further comprises a bottom anti-skid pad including a resilient, flexible base.

20. The patient positioning support of claim 13 wherein said torso pad and said resilient, flexible arm restraints are about ⅝^{th} of an inch thick having a density of about 1.25 pounds per cubic foot to about 1.35 pounds per cubic foot and an indentation force deflection of about 37 pounds to about 43 pounds at 25 percent (25%) deflection to about 85 pounds to 91 pounds at 65 percent (65%) deflection.

21. The patient positioning support of claim 19 wherein said lower surface of said substantially flat flexible layer of the bottom anti-skid pad or member has a coefficient of static friction sufficient to prevent the torso pad from sliding or moving on the upper surface of the tiltable medical procedures table.

22. The patient positioning support of claim 21 wherein said bottom layer of anti-skid foam is preferably constructed of latex-free, hydrophilic tight cell polyurethane foam.
